(19) United States
(12) Patent Application Publication
(10) Pub. No.: US 2005/0065509 A1
(43) Pub. Date: Mar. 24, 2005

(54) Flat electrode arrays for generating flat lesions

(75) Inventors: Douglas M. Coldwell, Philadelphia, PA (US); Robert F. Rioux, Ashland, MA (US)

Correspondence Address: Bingham McCuthen, LLP;Suite 1800, Three Embarcadero, San Francisco, CA, 94111-4067, US

(73) Assignee: SciMed Life Systems, Inc., Maple Grove, MN (US)

(21) Appl. No.: 10/668,995

(22) Filed: Sep. 22, 2003

(51) Int. Cl. A61B 18/14

(52) U.S. Cl. 606/41

(57) ABSTRACT

Apparatus for treating pleurae with electrical energy includes a cannula including proximal and distal ends defining a longitudinal axis therebetween, and an array of electrodes disposed within a lumen of the cannula and deployable from the distal end of the cannula. The electrodes may extend in a direction substantially perpendicular to the longitudinal axis when deployed from the cannula, thereby defining a plane. During use, a pleura to be treated may be exposed or the cannula may be inserted into a thoracic cavity until the distal end is adjacent the pleura. The electrodes are advanced from the cannula such that distal portions of the electrodes extends away from one another and lie within a plane. The distal portions are placed in contact with the pleura, and electrical energy is delivered from the electrodes to treat the pleura, e.g., to ablate cancerous tissue and/or cause coagulation.

### FIELD OF INVENTION

The present invention relates generally to apparatus and methods for treating tissue using electrical energy, and more particularly, to apparatus and methods for creating lesions in or on a surface of a tissue structure, for example, for generating pleural lesions in a patient's lung.

### BACKGROUND

Tissue may be destroyed, ablated, or otherwise treated using thermal energy during various therapeutic procedures. For example, electrical energy may be used to treat patients with tissue anomalies, such as cancerous or benign tumors within a liver, stomach, bowel, pancreas, kidney, or lung. Undesirable cells may be destroyed using heat generated by delivering the electrical energy, e.g., within the radio frequency (“RF”) range, into the tissue to be treated.

Various RF ablation devices have been suggested for this purpose. For example, U.S. Pat. No. 5,855,576 discloses ablation apparatus that include a plurality of wire electrodes deployable from a cannula or catheter. Each of the electrodes includes a proximal end that is coupled to a generator, and a distal end that may be advanced from a distal end of the cannula. When advanced, the electrodes may extend in a continuous curve from the cannula, e.g., into an umbrella-like array with the distal ends located generally radially and uniformly spaced apart from the catheter distal end.

The electrodes may be energized in a monopolar or bipolar configuration to heat and/or necrose tissue within a precisely defined volumetric region of target tissue. The electrical energy may be delivered in a bipolar mode (between multiple active electrodes), or in a monopolar mode (between one or more active electrodes and one or more dispersive electrodes located remotely from the tissue being treated).

Accordingly, apparatus and methods for treating tissue using electrical energy would be useful.

### SUMMARY OF INVENTION

The present invention is directed to apparatus and methods for creating lesions in a surface of a tissue structure, for example, for generating pleural lesions in a patient's lung.

In accordance with one aspect of the invention, an apparatus is provided for treating tissue with electrical energy that includes an elongate cannula including proximal and distal ends defining a longitudinal axis therebetween, and an array of electrodes disposed within a lumen of the cannula and deployable from the distal end of the cannula. Preferably, the electrodes extend in a direction that is substantially perpendicular to the longitudinal axis when deployed from the cannula, thereby defining a plane. More preferably, the electrodes are substantially flat tines lying within the plane when the electrodes are deployed from the cannula and/or the electrodes may terminate in substantially blunt distal tips.

In accordance with another aspect of the invention, a method is provided for treating a tissue structure using a cannula including an array of electrodes deployable from a distal end thereof. The electrodes may be advanced from the distal end of the cannula such that distal portions of the electrodes lie substantially within a plane. The distal portions of the electrodes may be placed in contact with a surface of the tissue structure, and electrical energy may be delivered from the electrodes into the surface to treat the tissue structure.

In one embodiment, the electrical energy may be delivered for sufficient time to create a lesion in the surface, e.g., to necrose tissue adjacent the surface. Alternatively, the electrical energy may be delivered for sufficient time to cause coagulation of the surface.

In accordance with still another aspect of the present invention, a method is provided for treating a pleura using a cannula including an array of electrodes deployable from a distal end thereof. The electrodes may be advanced from the distal end of the cannula such that distal portions of the electrodes lie substantially within a plane. The distal portions of the electrodes may be placed in contact with the pleura, and electrical energy may be delivered from the distal portions of the electrodes to treat the pleura.

The pleura may be exposed before placing the distal portions of the electrodes in contact with the pleura, e.g., using conventional thoracic access, such as a thoracotomy or sternotomy. Alternatively, the cannula may be advanced through a port, other minimally invasive opening, or directly into the chest with the electrodes retracted within the cannula. Once the distal end of the cannula is disposed within the thoracic cavity, the electrodes may be deployed and placed in contact with the surface of the pleura being treated.

For example, the pleura may include cancerous tissue, and the electrical energy may be delivered for sufficient time to necrose at least a portion of the pleura, e.g. to destroy all or a portion of the cancerous tissue. Alternatively, the energy may be applied for sufficient time to cause coagulation without causing necrosis of tissue adjacent the electrodes. Optionally, the electrodes may be reapplied to multiple areas of the surface to treat a target portion of the pleura.

Other aspects and features of the invention will become apparent from consideration of the following description taken in conjunction with the accompanying drawings.

### BRIEF DESCRIPTION OF THE DRAWINGS

The drawings illustrate the design and utility of preferred embodiments of the invention, in which similar elements are referred to by common reference numerals, and in which:

FIG. 1 is a partial cross-sectional view of a patient's body, showing the patient's chest cavity.

FIG. 2 is a perspective view of a preferred embodiment of an apparatus including a plurality of electrodes deployed from a cannula.

FIGS. 3A and 3B are cross-sectional side views of the apparatus of FIG. 2, with the electrodes retracted into and deployed from the cannula, respectively.

FIGS. 4A-4C are cross-sectional views of a chest cavity, showing a method for treating a pleura therein, in accordance with the present invention.

### DETAILED DESCRIPTION OF THE ILLUSTRATED EMBODIMENTS

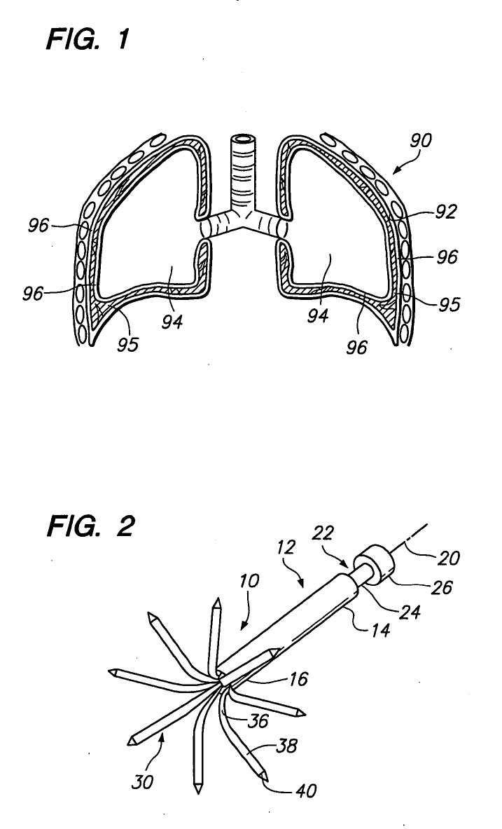

Turning to the drawings, FIG. 1 is a cross-sectional view of a chest cavity 92 of a patient 90, including lungs 94 disposed within pleurae 96. Each pleura 96 is a thin membrane of moist tissue that surrounds a lung 94 and/or provides a lining for the chest cavity 92, thereby defining a pleural space 95 within the chest cavity 92, but outside each lung 94. Normally, the lung 94 and chest wall are separated only by the pleurae 96, and the pleural space 95 is relatively small. If air or other fluid enters the pleural space 95, it may cause the lung 94 to collapse.

A pleural effusion is an abnormal collection of fluid within the pleura 96. Transudative pleural effusions may be caused by a disorder in the normal pressure in the lung, as may occur in patients with congestive heart failure. In addition, exudative pleural effusions may be caused by disease within the lung 94, such as cancer, tuberculosis, drug reactions, lung infections, asbestosis, sarcoidosis, and the like.

The apparatus and methods of the present invention may be used for treating mesothelioma, a cancer of the pleura, or other conditions of the pleurae. One or more pleurae may be treated alone or in conjunction with other procedures, e.g., surgery or other procedures involving the lungs. In addition or alternatively, the apparatus and methods described herein may also be used for treating other tissue structures, e.g., liver, kidney, pancreas, and the like, to cause coagulation, tissue necrosis, and the like. By way of non-limiting example, the apparatus and methods described herein may also be used to treat peritoneal carcinomatosis (spread of cancer to the peritoneal lining of the abdomen).

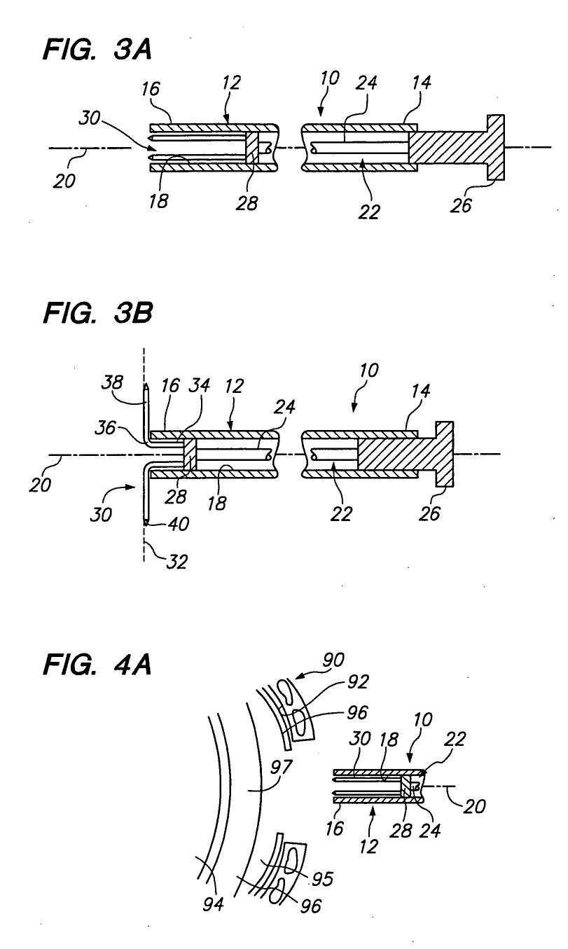

Turning to FIGS. 2, 3A, and 3B, a preferred embodiment of an apparatus 10 for treating tissue with electrical energy may include a cannula 12, and a plurality of electrodes 30 deployable from the cannula 12. The cannula 12 may be a substantially rigid tubular member including a proximal end 14, a distal end 16, and a lumen 18 extending therebetween, thereby defining a longitudinal axis 20. Alternatively, the cannula 12 may be semi-rigid, flexible, and/or malleable, if desired. Optionally, the cannula 12 may include a handle or other structures (not shown) on the proximal end 14, e.g., to facilitate manipulating and/or stabilizing the apparatus 10 during use.

The cannula 12 may be formed at least partially from an electrically insulating material, e.g., plastic, and/or an electrically conductive material, e.g., stainless steel or other metal, covered with an electrically insulating coating or sleeve. Thus, the cannula 12, particularly the proximal end 14, may be electrically isolated from the electrodes 30 to ensure safe use of the apparatus 10.

The cannula 12 may have a substantially blunt distal end 16, as shown, although, alternatively, the distal end 16 may include a sharpened distal tip (not shown) that may be penetrated directly into tissue. Notably, the distal tips of the electrodes 30 are sharpened in order to easily penetrated directly into and through tissue. Thus, the cannula 12 and electrodes 30 may be similar to cannulas used in the LeVeen™ Needle Electrode or CoAccess™ Electrode, available from Boston Scientific Meditech, San Jose, Calif. Additional information on these cannulas may be found in U.S. Pat. Nos. 5,868,740, 6,050,992 and 6,337,998, the disclosures of which are expressly incorporated by reference herein.

As best seen in FIGS. 3A and 3B, a plunger or other structure 22 may be provided within the lumen 18 for deploying the electrodes 30 from and/or retracting the electrodes 30 into the lumen 18 of the cannula 12. For example, the plunger 22 may include an elongate shaft 24 extending from a handle 26 into the lumen 18 of the cannula 12. The shaft 24 may terminate in a piston 28 slidably disposed within the lumen 18 to which the electrodes 30 may be attached. Detents or other elements (not shown) may be provided on the cannula 12 and/or plunger 22 for limiting movement of the plunger 22 relative to the cannula 12, as are well known in the art. For example, a cooperating set screw and slot may be provided to prevent the plunger 22 from being removed proximally from the cannula 12.

The shaft 24 may be sufficiently rigid to prevent buckling, e.g., when the plunger 22 is advanced relative to the cannula 12, as during deployment of the electrodes 30. The shaft 24 may include one or more wires or other electrical leads (not shown) therein that may be coupled to the electrodes 30 for delivering electrical energy from a source of energy, e.g., a radio frequency (“RF”) generator (not shown), to the electrodes 30. One or more electrical connectors (also not shown) may be provided on the plunger 22, e.g., on the handle 26 that may be coupled to the electrical lead(s).

Thus, one or more cables (not shown) may be connected to the apparatus 10, via the one or more connectors, that may be coupled to an energy source for delivering electrical energy to the electrodes 30. For example, if the apparatus 10 were a monopolar device, a single connector (not shown) may be provided on the handle 26 such that a cable (also not shown) may be connected to the connector for coupling the electrodes 30 to an output terminal of a RF generator (also not shown).

Returning to FIGS. 2, 3A, and 3B, the electrodes 30 may be retracted within the lumen 18 of the cannula 12, thereby defining a contracted condition, as shown in FIG. 3A, and advanced from the distal end 16 of the cannula 12, thereby defining an expanded condition, as shown in FIGS. 2 and 3B. In a preferred embodiment, in the expanded condition, the electrodes 30 may extend in a direction substantially perpendicular to the longitudinal axis 20 of the cannula 12, thereby substantially defining a plane 32.

Each electrode 30 may include a proximal portion 34, an intermediate portion 36, and a distal portion 38, which may have similar or different widths and/or thicknesses than one another, depending upon the desired mechanical properties of the electrode 30. For example, the proximal and distal portions 34, 38, may be substantially straight, and the intermediate portion 36 may be biased to curve in a desired manner. Preferably, the curvature of the intermediate portion 36 is such that, when the electrodes 30 are extended from the cannula 12, the distal portions 38 may move outwardly away from one another until they lie substantially within the plane 32. For example, the intermediate portions 36 may be biased to create a ninety degree (90°) curve.

The proximal portions 34 may remain substantially within the lumen 18 of the cannula 12, even when the electrodes 30 are deployed. The proximal portions 34 may be mechanically attached to the piston 28, e.g., using an adhesive, interference fit, cooperating connectors, welding, and the like, to substantially permanently fix the electrodes 30 to the plunger 22. In addition, the proximal portions 34 may be electrically coupled to the one or more leads within the plunger 22 to deliver electrical energy to the distal portions 38 of the electrodes 30.

Each electrode 30 may be a solid or hollow wire, a band or strip of material, and the like, formed at least partially from an electrically conductive material, thereby providing an elongate tine. For example, each electrode 30 may be formed from an elastic spring material, e.g., stainless steel, and/or a superelastic material, e.g., Nitinol. Thus, the electrodes 30 may be elastically constrained when retracted into the cannula 12, but may automatically expand upon deployment, returning substantially to the expanded configuration shown in FIGS. 2 and 3B.

Preferably, the entire length of each electrode 30 is electrically conductive such that any portion of the electrodes 30 that is exposed from the cannula 12, e.g., the distal portions 38, may be used to deliver electrical energy. Alternatively, an insulating sleeve or coating may be provided on a portion of the electrodes 30, e.g., the proximal portion 34, the intermediate portion 36, and/or at least partially on the distal portion 38.

In a preferred embodiment, each electrode 30 is a substantially flat band, e.g., having a width between about 0.20-0.40 mm, a thickness between about 0.10-0.20 mm, with a nominal width and thickness of 0.32 mm and 0.16 mm, respectively, and an overall length between about 100-250 mm. The distal portions 38 may have a length between about 10-30 mm, such that the electrodes 30 generally define a circle when deployed, as best seen in FIG. 2. Thus, the electrodes 30 may be used to treat an area at least as larger as the area of the circle defined by the distal portions 38, as explained further below. The electrodes 30 may be arranged such that, when deployed, the width of the electrodes 30 lie generally within the plane 32, thereby maximizing contact.

Optionally, the distal portion 38 of each electrode 30 may terminate in a substantially blunt and/or rounded distal tip 40, as shown in FIG. 2. Alternatively, the distal portion 38 may terminate in a sharpened distal tip that may be penetrated directly into tissue.

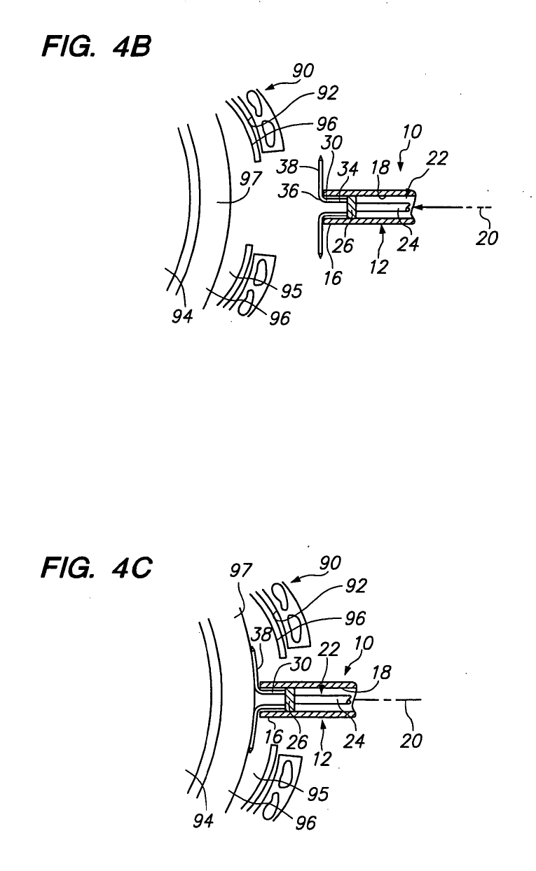

Turning to FIGS. 4A-4C, during use, an apparatus 10, such as that described above, may be used to treat pleural tissue 97 within a patient's chest 92. As shown in FIGS. 3A and 4A, the apparatus 10 may be provided initially with the electrodes 30 retracted into the cannula 12.

The pleura 96 to be treated may be exposed using an open surgical approach, such as a sternotomy or thoracotomy, which is well known to those skilled in the art. Alternatively, a port or other minimally invasive opening (not shown) may be used to access the chest cavity 92. In a further alternative, if the cannula 12 includes a sharpened distal end (not shown), the cannula 12 may be inserted directly into tissue, e.g., through the patient's skin and any intervening tissue (not shown) until the cannula 12 enters the chest cavity 92.

Once the pleura 96 is exposed or the cannula 12 is disposed within the chest cavity 92, the electrodes 30 may be advanced from the cannula 12, e.g., such that the distal portions 38 of the electrodes 30 lie substantially within a plane, as shown in FIG. 4B. This may involve pushing the handle 26 (not shown, see FIG. 2) to advance the plunger 22, as explained previously.

Turning to FIG. 4C, with the distal portions 38 of the electrodes 30 deployed, the electrodes 30 may be placed in contact with the surface of the pleural tissue 97 to be treated. Sufficient pressure may be applied to ensure substantial contact between the electrodes 30 and the pleural tissue 97, e.g., to minimize gaps or discontinuities in tissue/electrode contact.

Electrical energy may then be delivered from the electrodes 30 to treat the pleural tissue 97. Preferably, energy is delivered in a monopolar mode. If so, one or more dispersive electrodes may be placed previously in contact with the patient's body, preferably at locations remote from the treatment site, e.g., the patient's legs and/or back (not shown), as is well known to those skilled in the art. Alternatively, energy may be delivered in a bipolar mode, e.g., by coupling one or more of the electrodes 30 to opposite terminals of a RF generator (not shown), as is well known to those skilled in the art.

The duration of energy delivery may vary depending upon the intended treatment. For example, if the pleura 96 includes cancerous tissue, energy may be delivered for sufficient time to cause necrosis of the pleural tissue 97 contacted by and/or underlying the electrodes 30. If a single placement and energy delivery do not destroy a large enough region of tissue, the apparatus 10 may be moved to another location, e.g., adjacent to the first location, and the procedure repeated as many times as necessary to destroy the target tissue 97. Alternatively, the apparatus 10 may be used simply to coagulate pleural tissue 97, e.g., to prevent bleeding at one or more locations along the pleura 96.

This procedure may be completed alone or in conjunction with other procedures being performed on the patient 90. For example, a tube or other device (not shown) may be introduced to evacuate fluid from within the pleural space 95 before or after treating the pleural tissue 97. In addition or alternatively, other procedures may be performed on the lung 94 underlying the pleura 96, e.g., tissue ablation, surgical resection, and the like.

Although the method described above is directed to treating pleural tissue, the apparatus and methods of the present invention may be suitable for ablating, coagulating, or otherwise treating tissue in other regions of the body. For example, tumors near the surface of an organ may be treated by placing the substantially flat array of electrodes against the surface of the organ overlying the tumor, and delivering energy, similar to the methods described above. Such an organ, such as a liver, kidney, pancreas, lung, and the like; may be surgically exposed using known procedures. The deployed flat array of electrodes may be placed against the organ and energy delivered without penetrating into the organ.

Blunt and/or rounded tips on the electrodes may be used on selected devices to protect against undesired punctures into the organ if such punctures are undesired. In addition or alternatively, flat band electrodes may increase surface contact with the tissue structure, thereby reducing the chance of localized charring.

Similar to the methods described above, the apparatus the present invention may be used as a surface coagulator for coagulating large surface areas of a tissue structure, as will be appreciated by those skilled in the art.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. An apparatus for treating tissue with electrical energy, comprising:
a substantially rigid cannula comprising proximal and distal ends defining a longitudinal axis therebetween, and a lumen extending between the proximal and distal ends;
an array of electrodes disposed'within the lumen of the cannula and deployable from the distal end of the cannula substantially perpendicular to the longitudinal axis, thereby substantially defining a plane.

2. The apparatus of claim 1, wherein the electrodes comprise substantially flat tines comprising a width lying generally within the plane when the electrodes are deployed from the cannula.

3. The apparatus of claim 1, wherein each of the electrodes comprises a distal portion that is substantially straight and an intermediate portion that is curved when the electrodes are deployed from the cannula such that each distal portions lies substantially within the plane when the electrodes are deployed from the cannula.

4. The apparatus of claim 1, wherein each of the electrodes terminates in a substantially blunt distal tip.

5. The apparatus of claim 1, wherein the cannula terminates in a substantially blunt distal tip.

6. The apparatus of claim 1, wherein the cannula terminates in a sharpened distal tip.

7. The apparatus of claim 1, wherein the electrodes are biased to extend in a direction substantially perpendicular to the longitudinal axis, the electrodes being deflectable into a compressed configuration when retracted into the lumen of the cannula.

8. A method for treating a tissue structure using a cannula comprising an array of electrodes deployable from a distal end thereof, the method comprising:
advancing the electrodes from the distal end of the cannula such that distal portions of the electrodes lie substantially within a plane;
placing the distal portions of the electrodes in contact with a surface of the tissue structure; and
delivering electrical energy from the electrodes to the surface to treat the tissue structure.

9. The method of claim 8, wherein the tissue structure comprises a pleura.

10. The method of claim 9, wherein the surface comprises a tumor.

11. The method of claim 10, further comprising exposing the pleura before placing the distal portions of the electrodes in contact with the surface of the pleura.

12. The method of claim 10, wherein the electrical energy is delivered for sufficient time to destroy at least a portion of the tumor.

13. The method of claim 8, wherein the electrical energy is delivered for sufficient time to create a lesion in the surface.

14. The method of claim 8, wherein the electrical energy is delivered for sufficient time to cause coagulation of the surface.

15. A method for treating a pleura using a cannula comprising a plurality of electrodes deployable from a distal end thereof, the method comprising:
advancing the plurality of electrodes from the distal end of the cannula such that distal portions of the electrodes lie substantially within a plane;
placing the distal portions of the electrodes in contact with the pleura; and
delivering electrical energy from the electrodes to treat the pleura.

16. The method of claim 15, further comprising exposing the pleura before placing the distal portions of the electrodes in contact with the pleura.

17. The method of claim 15, wherein the pleura comprises cancerous tissue.

18. The method of claim 15, wherein the electrical energy is delivered for sufficient time to cause necrosis of at least a portion of the pleura.

19. The method of claim 15, wherein the electrical energy is delivered for sufficient time to cause coagulation of the pleura.

20. The method of claim 15, further comprising inserting the distal end of the cannula into a thoracic cavity before advancing the plurality of electrodes from the distal end of the cannula.
